(19) United States
(12) Patent Application Publication
(10) Pub. No.: US 2003/0032172 A1
(43) Pub. Date: Feb. 13, 2003

(54) Automated nucleic acid assay system

(75) Inventors: Billy W. Colston JR., Livermore, CA (US); Steve B. Brown, Livermore, CA (US); Shanavaz L. Nasarabadi, Livermore, CA (US); Phillip Belgrader, Manteca, CA (US); Fred Milanovich, Lafayette, CA (US); Graham Marshall, Fox Island, WA (US); Don Olson, Gig Harbor, WA (US); Duane Wolcott, Fox Island, WA (US)

Correspondence Address: Eddie E. Scott, P.O. Box 808, L-703, Livermore, CA, 94551, US

(73) Assignee: The Regents of the University of California

(21) Appl. No.: 10/189,319

(22) Filed: Jul. 2, 2002

Related U.S. Application Data
(60) Provisional application No. 60/303,637, filed on Jul. 6, 2001.

(51) Int. Cl. C12M 1/38; C12Q 1/68

(52) U.S. Cl. 435/287.2; 435/286.5; 435/6; 435/91.2

(57) ABSTRACT

A nucleic acid assay system includes a holding means that receives a sample and a reagent. A PCR reactor means amplifies the sample and produces an amplified sample. A detection means detects PCR amplicon. A transport means selectively transports the sample and the reagent relative to the holding means, the PCR reactor means, and the detection means. A control means is provided for selectively adding the reagent to the sample, mixing the sample and the reagent, performing PCR amplification, and detecting PCR amplicon. A decontamination means is provided for decontaminating the holding means, the PCR reactor means, and the detection means.

### CROSS-REFERENCE TO RELATED APPLICATIONS

This application claims the benefit of U.S. Provisional Application No. 60/303,537, filed Jul. 6, 2001, and titled “AUTOMATED NUCLEIC ACID ASSAY SAMPLE PREPARATION AND DETECTION,” which is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND OF THE INVENTION

1. Field of Endeavor

The present invention relates to an assay system and more particularly to a nucleic acid assay system.

2. State of Technology

U.S. Pat. No. 3,241,432 to Leonard T. Skeggs, et al., issued Mar. 22, 1966 and U.S. Pat. No. 3,604,814 issued Sep. 14, 1971 to Leonard T. Skeggs describe systems for analysis of fluid samples that have gained remarkably wide commercial acceptance through the extremely rapid and reliable operation.

U.S. Pat. No. 4,022,575 for an automatic chemical analyzer to Elo H. Hansen and Jaromir Ruzicka issued May 10, 1977 provide the following background information, “The ever increasing demand for numbers of analyses in clinical, agricultural, pharmaceutical and other types of analytical control has lead to the development of a large number of various instruments for automated analysis. The development in this field is further being stimulated by the additional advantages gained by automation: increased precision, decreased cost per assay and good reliability of the automated equipment.”

U.S. Pat. No. 4,315,754 for flow injection analysis with intermittent flow to Jaromir Ruzicka and Elo H. Hansen issued Feb. 16, 1982 provides the following background information, “Flow injection analysis, FIA, has opened up new areas within the field of analysis. FIA is a continuous analysis system in which discrete volumes of sample solution are successively injected into a continuous, unobstructed carrier stream. The sample solutions react with the carrier stream and a detector for registering the results of the reactions is placed downstream from the point of injection.”

U.S. Pat. No. 5,589,136 for silicon-based sleeve devices for chemical reactions, assigned to the Regents of the University of California, inventors: M. Allen Northrup, Raymond P. Mariella, Jr., Anthony V. Carrano, and Joseph W. Balch, patented Dec. 31, 1996 provides the following background information:

“Current instruments for performing chemical synthesis through thermal control and cycling are generally very large (table-top) and inefficient, and often they work by heating and cooling of a large thermal mass (e.g., an aluminum block). In recent years efforts have been directed to miniaturization of these instruments by designing and constructing reaction chambers out of silicon and silicon-based materials (e.g., silicon, nitride, polycrystalline silicon) that have integrated heaters and cooling via convection through the silicon.

Microfabrication technologies are now well known and include sputtering, electrodeposition, low-pressure vapor deposition, photolithography, and etching. Microfabricated devices are usually formed on crystalline substrates, such as silicon and gallium arsenide, but may be formed on non-crystalline materials, such as glass or certain polymers. The shapes of crystalline devices can be precisely controlled since etched surfaces are generally crystal planes, and crystalline materials may be bonded by processes such as fusion at elevated temperatures, anodic bonding, or field-assisted methods.

Monolithic microfabrication technology now enables the production of electrical, mechanical, electromechanical, optical, chemical and thermal devices, including pumps, valves, heaters, mixers, and detectors for microliter to nanoliter quantities of gases, liquids, and solids. Also, optical waveguide probes and ultrasonic flexural-wave sensors can now be produced on a microscale. The integration of these microfabricated devices into a single system allows for the batch production of microscale reactor-based analytical instruments. Such integrated microinstruments may be applied to biochemical, inorganic, or organic chemical reactions to perform biomedical and environmental diagnostics, as well as biotechnological processing and detection.

The operation of such integrated microinstruments is easily automated, and since the analysis can be performed in situ, contamination is very low. Because of the inherently small sizes of such devices, the heating and cooling can be extremely rapid. These devices have very low power requirement and can be powered by batteries or by electromagnetic, capacitive, inductive or optical coupling.

The small volumes and high surface-area to volume ratios of microfabricated reaction instruments provide a high level of control of the parameters of a reaction. Heaters may produce temperature cycling or ramping; while sonochemical and sonophysical changes in conformational structures may be produced by ultrasound transducers; and polymerizations may be generated by incident optical radiation.

Synthesis reactions, and especially synthesis chain reactions such as the polymerase chain reaction (PCR), are particularly well-suited for microfabrication reaction instruments. PCR can selectively amplify a single molecule of DNA (or RNA) of an organism by a factor of 10.sup.6 to 10.sup.9. This well-established procedure requires the repetition of heating (denaturing) and cooling (annealing) cycles in the presence of an original DNA target molecule, specific DNA primers, deoxynucleotide triphosphates, and DNA polymerase enzymes and cofactors. Each cycle produces a doubling of the target DNA sequence, leading to an exponential accumulation of the target sequence.

The PCR procedure involves: 1) processing of the sample to release target DNA molecules into a crude extract; 2) addition of an aqueous solution containing enzymes, buffers deoxyribonucleotide triphosphates (dNTPS), and aligonucleotide primers; 3) thermal cycling of the reaction mixture between two or three temperatures (e.g., 90. degree.-96.degree., 72.degree., and 37.degree.-55.degree. C.); and 4) detection of amplified DNA. Intermediate steps, such as purification of the reaction products and the incorporation of surface-bending primers, for example, may be incorporated in the PCR procedure.

A problem with standard PCR laboratory techniques is that the PCR reactions may be contaminated or inhibited by the introduction of a single contaminant molecule of extraneous DNA, such as those from previous experiments, or other contaminants, during transfers of reagents from one vessel to another. Also, PCR reaction volumes used in standard laboratory techniques are typically on the order of 50 microliters. A thermal cycle typically consists of four stages: heating a sample to a first temperature, maintaining the sample at the first temperature, cooling the sample to a second lower temperature, and maintaining the temperature at that lower temperature. Typically, each of these four stages of a thermal cycle requires about one minute, and thus to complete forty cycles, for example, is about three hours. Thus, due to the large volume typically used in standard laboratory procedures, the time involved, as well as the contamination possibilities during transfers of reagents from one vessel to another, there is clearly a need for microinstruments capable of carrying out the PCR procedure.”

### SUMMARY OF THE INVENTION

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a nucleic acid assay system for analyzing a sample using a reagent. A holding means receives the sample and the reagent. A PCR reactor means amplifies the sample and produces an amplified sample. A detection means detects PCR amplicon. A transport means selectively transports the sample, the reagent, and the amplified sample relative to the holding means, the PCR reactor means, and the detection means. The transport means is operatively connected to the holding means, the PCR reactor means, and the detection means. Control means is provided for selectively adding the reagent to the sample, mixing the sample and the reagent, performing PCR amplification, and detecting PCR amplicon. The control means is operatively connected to the holding means, the PCR reactor means, the detection means, and the transport means. A decontamination means is provided for decontaminating the holding means, the PCR reactor means, and the detection means.

One embodiment of the invention provides a nucleic acid assay method comprising a number of steps. One step comprises automatically injecting and or aspirating a sample. Another step comprises automatically adding PCR reagent to the sample. Another step comprises automatically mixing the sample and the reagent. Another step comprises automatically transporting the sample and the reagent to a PCR reactor. The PCR reactor consisting of a fluidics system. Another step comprises automatically performing PCR amplification, resulting in an amplified sample. Another step comprises automatically transporting the amplified sample from the PCR reactor. Another step comprises automatically detecting PCR amplicon. In another step a decontamination means is provided for decontaminating the holding means, the PCR reactor means, and the detection means.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1A illustrates a system for performing autonomous, in-line nucleic acid sample preparation, amplification, and/or analysis.

FIG. 1B illustrates another embodiment of a system for performing autonomous, in-line nucleic acid sample preparation, amplification, and/or analysis.

FIG. 2 illustrates another system for performing autonomous, nucleic acid assay.

FIG. 3 illustrates yet another embodiment of a system for performing autonomous, nucleic acid assay.

FIG. 4 illustrates a system representing another embodiment of the present invention.

FIG. 5A illustrates a system for performing autonomous, in-line nucleic acid sample preparation, amplification, and/or analysis.

FIG. 5B illustrates another embodiment of a system for performing autonomous, in-line nucleic acid sample preparation, amplification, and/or analysis.

FIG. 6 illustrates yet another embodiment of a system for performing autonomous, nucleic acid assay.

### DETAILED DESCRIPTION OF THE INVENTION

Referring now to the drawings, to the following detailed information, and to incorporated materials; a detailed description of the invention, including specific embodiments, is presented. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

Nucleic acid amplification and detection is a widely used technique for conducting biological research. Utilization is applied to an increasing range of applications including diagnostics in bench-top research to the clinical arena, genomic screening for drug discovery to toxicology, screening for contamination to identification. Conventional sample preparation and analysis techniques for performing nucleic acid assays are time-consuming, require trained technicians, and lack precise repeatability. New technical developments are needed to improve the performance of nucleic acid amplification and detection.

Zone fluidics defines a general-purpose fluidics tool, allowing the precise manipulation of gases, liquids and solids to accomplish very complex analytical manipulations with relatively simple hardware. Zone fluidics is the precisely controlled physical, chemical, and fluid-dynamic manipulation of zones of miscible and immiscible fluids in narrow bore conduits to accomplish sample conditioning and chemical analysis. A zone is a volume region within a flow conduit containing at least one unique characteristic.

A unit operation in zone fluidics comprises of a set of fluid handling steps intended to contribute to the transformation of the sample into a detectable species or prepare it for manipulation in subsequent unit operations. Examples of unit operations include sample filtering, dilution, enrichment, medium exchange, headspace sampling, solvent extraction, matrix elimination, de-bubbling, amplifying, hybridizing, and reacting. In current analytical practice many of these steps are handled manually or in isolated pieces of equipment. Integration is scant at best, and there is a high degree of analyst involvement. In zone fluidics, sample and reagent zones are subjected to these unit operations in a sequential manner being transported from one unit operation to the next under fluidic control.

Early attempts to automate analytical science turned to robotics, but the high cost of instrumentation and excessive complexity demanded large budgets both in terms of hardware and research effort. With the rapid growth in genomics, and proteomics, and high throughput screening techniques, robotics has enjoyed resurgence. The requirement for large hardware budgets and research resources has not changed.

Zone fluidics proposes an alternative approach whereby unit operations are performed in narrow bore conduits and the transportation medium, instead of being mechanical as in robotics, is fluidic. At the heart of a zone fluidics manifold is a multi-position selection valve. Fluids are propelled and manipulated in the manifold by means of a bi-directional flow pump. A holding coil between the pump and valve is used to stack zones and mix adjacent zones through dispersion and diffusion as is practiced in sequential injection analysis (SIA).

The ports of the multi-position valve are coupled to various reservoirs, reactors, manifold devices, and detectors as indicated. Narrow bore conduits comprise the flow channels and provide fluid contact between manifold devices and components. (The term fluid refers to liquids, gases, aerosols, and suspensions.)

Samples in zone fluidics are not limited to liquids. Rather, gases, and suspensions containing solids or cells are also included. Where solid samples are used, particles are limited to a size that ensures no blockages.

In most cases, reagents are prepared and then coupled to the zone fluidics manifold. The metering capability of the pump and mixing unit operations allow for reagents and standards to be prepared in situ. Reagents can therefore be presented to the zone fluidics manifold in an appropriately designed cartridge as ready-made, reagent concentrates, lyophilized, or crystalline form. Standards can be plumbed to the multi-position valve as discrete reservoirs providing the required range of concentrations. As for reagents though, standards can also be prepared in situ or diluted to cover a larger dynamic range.

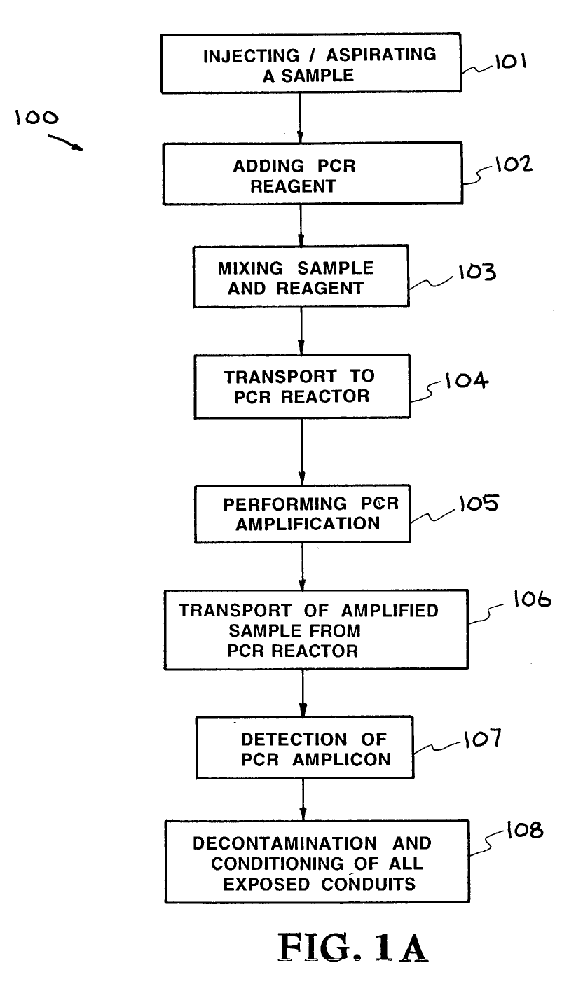

Referring now to FIG. 1, a system for performing autonomous, nucleic acid assay is illustrated. The system is generally designated by the reference numeral 100. The system 100 provides a system capable of performing, singly or in combination, sample preparation, nucleic acid amplification, and nucleic acid detection functions. Some of the uses of the nucleic acid assay system 100 are: biowarfare detection applications including identifying, detecting, and monitoring bio-threat agents that contain nucleic acid signatures, such as spores, bacteria, etc.; biomedical applications including tracking, identifying, and monitoring outbreaks of infectious disease and automated processing, amplification, and detection of host or microbial DNA in biological fluids for medical purposes; forensic applications including automated processing, amplification, and detection DNA in biological fluids for forensic purposes; and food and beverage safety including automated food testing for bacterial contamination.

The nucleic acid assay system 100 includes a number components. A means 101 for injecting and or aspirating a sample provides injection and/or aspiration of the sample. In one embodiment the injecting/aspirating means 101 consists of a zone fluidics system. In another embodiment the injecting/aspirating means 101 consists of an FIA system. The means 101 for injecting and or aspirating a sample can be, for example, a injecting/aspirating device available under the trademark milliGAT™ pump, Global FIA, Inc, Fox Island, Wash.

A means 102 for adding PCR reagent to the sample is operatively connected to the means 101 for injecting and or aspirating a sample. The means 102 for adding PCR reagent to the sample can be, for example, a unit for adding PCR reagent to the sample such as an injection or multi position selection valve, available from VICI, Houston, Tex.

A means 103 for mixing the sample and the reagent is operatively connected to the means 102 for adding PCR reagent to the sample. The mixing means 103 mixes the sample with a PCR reagent. In one embodiment the PCR reagent includes primers. In another embodiment the PCR reagent includes oligos. The means 103 for mixing the sample and the reagent can be, for example, a super serpentine reactor, available from Global FIA, Inc, Fox Island, Wash.

A means 104 for transporting the sample and the reagent to a PCR reactor is operatively connected to the means 103 for mixing the sample and the reagent. The means 104 for transporting the sample and the reagent to a PCR reactor consists of a fluidics system. The means 104 for transporting the sample and the reagent to a PCR reactor can be, for example, FEP tubing available from Cole-Parmer, Vernon Hills, Ill.

A means 105 for performing PCR amplification is operatively connected to the means 104 for transporting the sample and the reagent to a PCR reactor. This results in an amplified sample. In one embodiment the PCR amplification means 105 includes an embedded thermocouple calibration conduit. PCR amplification devices are described in publications such as U.S. Pat. No. 5,589,136 for silicon-based sleeve devices for chemical reactions, assigned to the Regents of the University of California, inventors: M. Allen Northrup, Raymond P. Mariella, Jr., Anthony V. Carrano, and Joseph W. Balch, patented Dec. 31, 1996 and many are commercially available such as ABI PRISM® 7700 Sequence Detection System by Applied Biosystems; iCycler iQ Real-Time PCR Detection System by Bio-Rad; and Smart Cycler® System by Cepheid.

A means 106 for transporting the amplified sample from the PCR reactor is operatively connected to the means 105 for performing PCR amplification. The means 106 for transporting the amplified sample from the PCR reactor can be, for example, FEP tubing available from Cole-Parmer, Vernon Hills, Ill.

A means 107 for detection of PCR amplicon is operatively connected to the means 106 for transporting the amplified sample from the PCR reactor. The means 107 for detection of PCR amplicon can be, for example, a detection system described in publications and products produced by Cepheid and Baltimore-based Environmental Technologies Group, Inc. (ETG), a part of London-based Smiths Aerospace.

Conduits are included within the means 101 for injecting and or aspirating a sample, means 102 for adding PCR reagent to the sample, means 103 for mixing the sample and the reagent, means 104 for transporting the sample and the reagent to a PCR reactor, means 105 for performing PCR amplification, means 106 for transporting the amplified sample from the PCR reactor, and means 107 for detection of PCR amplicon. A means 108 for decontamination and conditioning the conduits is directly connected to the means 107 for detection of PCR amplicon. The means 108 for decontamination and conditioning the conduits is operatively connected to the means 101 for injecting and or aspirating a sample, means 102 for adding PCR reagent to the sample, means 103 for mixing the sample and the reagent, means 104 for transporting the sample and the reagent to a PCR reactor, means 105 for performing PCR amplification, means 106 for transporting the amplified sample from the PCR reactor, and means 107 for detection of PCR amplicon. The decontamination and conditioning of all exposed conduits can be, for example, be performed by using a decontaminant, such as bleach, which is pumped through the exposed conduits and then washed from the system with a suitable wash solution.

Referring now to FIG. 1B another embodiment of a system for performing autonomous, in-line nucleic acid sample preparation, amplification, and/or analysis is illustrated. The system is generally designated by the reference numeral 150. The system 150 illustrates another embodiment of an amplification cell. The system 150 is an amplification system that is coupled to units such as units 101, 102, 103, and 104 of FIG. 1A. The system 150 includes a means for performing PCR amplification 151 and a means for detection of PCR amplicon 152 operatively connected to the means for performing PCR amplification 151. The detection is performed within the PCR reactor. The system 150 results in an amplified sample and detection of PCR amplicon is performed on the amplified sample. In one embodiment the PCR amplification means 151 includes an embedded thermocouple calibration conduit.

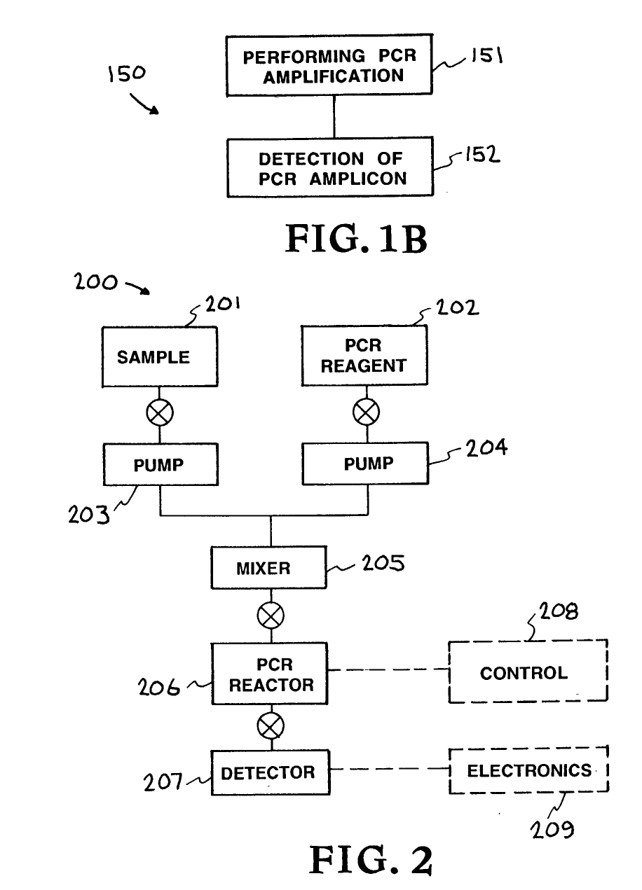

Referring now to FIG. 2, another system for performing autonomous, nucleic acid assay is illustrated. The system is generally designated by the reference numeral 200. The system 200 provides a system capable of performing, singly or in combination, sample preparation, nucleic acid amplification, and nucleic acid detection functions. The nucleic acid assay system 200 includes a number components. A sample is contained in unit 201. A PCR reagent is contained in unit 202. A pump 203 transfers the sample from unit 201 into mixer 205. A pump 204 transfers the PCR reagent from unit 202 into mixer 205.

The mixer 205 combines the sample and the PCR reagent. In one embodiment the PCR reagent includes primers. In another embodiment the PCR reagent includes oligos. The mixer 205 can be, for example, a super serpentine reactor, available from Global FIA, Inc, Fox Island, Wash.

The mixed sample and reagent are transferred to a PCR reactor 206. This results in an amplified sample. In one embodiment the PCR reactor 206 includes an embedded thermocouple calibration conduit. PCR amplification devices are described in publications such as U.S. Pat. No. 5,589,136 for silicon-based sleeve devices for chemical reactions, assigned to the Regents of the University of California, inventors: M. Allen Northrup, Raymond P. Mariella, Jr., Anthony V. Carrano, and Joseph W. Balch, patented Dec. 31, 1996 and many are commercially available such as ABI PRISM® 7700 Sequence Detection System by Applied Biosystems; iCycler iQ Real-Time PCR Detection System by Bio-Rad; and Smart Cycler® System by Cepheid.

The amplified sample is transferred from the PCR reactor 206 detector 207. The detector can be, for example, a detection system described in publications and products produced by Cepheid and Baltimore-based Environmental Technologies Group, Inc. (ETG), a part of London-based Smiths Aerospace.

The control unit 208 and electronics 209 are connected to PCR Reactor 206 and Detector 207 respectively. The control and electronics can also be included in units 206 and 207.

The systems 100, 150, and 200 provide nucleic acid assay systems and methods. The methods include a number of steps. One step consists of automatically injecting and or aspirating a sample. Another step consists of automatically adding PCR reagent to the sample. Another step consists of automatically mixing the sample and the reagent. Another step consists of automatically transporting the sample and the reagent to a PCR reactor. The PCR reactor consists of a fluidics system. Another step consists of automatically performing PCR amplification resulting in an amplified sample. Another step consists of automatically transporting the amplified sample from the PCR reactor. Another step consists of automatically detecting PCR amplicon. The method is performed in a nucleic acid assay system and the nucleic acid assay system is decontaminated and conditioned before a new sample is analyzed. The systems including both real time and post-PCR detection. The systems 100, 150, and 200 are ideal for monitoring type systems, such as those currently being developed to detect terrorist releases of aerosolized bioagents. On-site detection systems for infectious diseases under development will need to incorporate sample preparation and analysis functions. The systems 100, 150, and 200 allow relatively unskilled personnel, such as early responders, to perform real-time field or point-of-care nucleic acid assays.

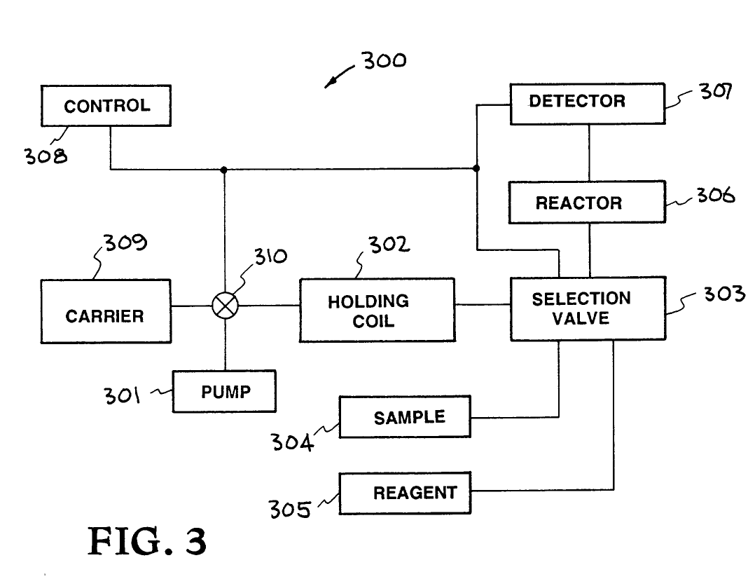

Referring now to FIG. 3, another embodiment of a system for performing autonomous, nucleic acid assay is illustrated. The system is generally designated by the reference numeral 300. The system 300 provides a system capable of performing, singly or in combination, sample preparation, nucleic acid amplification, and nucleic acid detection functions. Some of the uses of the nucleic acid assay system 300 are: biowarfare detection applications including identifying, detecting, and monitoring bio-threat agents that contain nucleic acid signatures, such as spores, bacteria, etc.; biomedical applications including tracking, identifying, and monitoring outbreaks of infectious disease and automated processing, amplification, and detection of host or microbial DNA in biological fluids for medical purposes; forensic applications including automated processing, amplification, and detection DNA in biological fluids for forensic purposes; and food and beverage safety including automated food testing for bacterial contamination.

The computer controlled zone fluidics system 300 performs sample preparation, sample delivery, sample isolation, and system decontamination functions. It is to be understood that multiple embodiments of zone fluidics system are envisioned. In the system 300, sample preparation and delivery is accomplished using a zone fluidics system including a pump 301, holding coil 302, selector valve 303, sample reservoir 304, and reagent reservoir 305. A reactor 306 and detector 307 are connected to the selection valve 303. A control unit 308 is operatively connected to the selection valve 303, the valve 310, pump 301, reactor 306, and detector 307. The control unit 308 and be a multipurpose computer or an individual control unit.

The pump 301 is used to draw and pump fluids into the holding coil 302. Fluids can be drawn from the sample unit 304 and the reagent unit 305. The carrier fluid unit 309 provides the medium for translating the pump movements into fluid handling actions. Aliquots of air or a hydrophobic liquid are used to spatially separate the carrier from reagent and sample volumes, greatly minimizing the chance of cross-contamination. The performance characteristics of the pump 301 allow for precise and accurate metering and positioning of aspirated zones in the flow manifold and flow cell. The holding coil 302 serves to mix various assay components (i.e., sample, oligonucleotides, primer, TaqMan probe, etc.) in preparation for amplification and/or detection. The holding coil 302 prevents contamination and is itself easily decontaminated by rinsing with buffer or some other cleaning agent (e.g., bleach). Nucleic acid amplification takes place in the reactor 306 once the pump 301 and control 308 have positioned the relevant components in the reactor 306. Nucleic acid detection and analysis takes place in the detector 307 once the pump 301 and control 308 have positioned the relevant components in the detector 307. The selection valve 303 serves as the interface between all components of the SIA unit, offering a flexible means of changing and upgrading the various fluidic components.

The nucleic acid amplification reactor 306 performs in-line amplification of the target DNA. This amplification is typically achieved using polymerase chain reaction (PCR) based methodologies, where a prepared sample and reagent mix is isolated and thermal cycling performed. This repeated heating and cooling of the mix selectively doubles a nucleic acid sequence during each thermal cycle. This process can occur in any thermal cycling type device that is amenable to PCR type amplification, including rapid micro-machined silicon type cyclers, block heater-based cyclers, etc. such as those designed by Idaho Tech. systems that use isothermic, enzyme regulated amplification can also be used.

Detection is detector 307 can occur either during (i.e., “real-time”) or after the amplification process. Real-time detection of amplified nucleic acid sequences is often preferable in field applications, because it does not require time-consuming post-PCR manipulation and processing. Examples of such post-PCR processes include slab gel and capillary electrophoresis, hybridization to immobilized oligonucleotides, or mass spectrometry. Real-time PCR can be accomplished using optical-based assays that either increase or decrease the emission from fluorescence-labeled probes during each amplification step. One commonly used technique for real-time PCR is TaqMan, a homogeneous PCR test that uses a fluorescence resonance energy transfer probe. This probe typically contains a “reporter” dye at the 5′ end and a “quencher” dye at the 3′ end. Intact, there is very little fluorescent emission from the probe, since the proximity of the quencher to the reporter dye serves to suppress the reporter emission. During PCR amplification, the probe anneals to a targeted complementary amplicon strand and begins extending one of the primers. An enzyme, (Taq polymerase) cleaves the probe and displaces both dye molecules, allowing them to separate and diffuse into the surrounding fluid. The resulting increase in reporter emission can be monitored and correlated PCR product concentration.

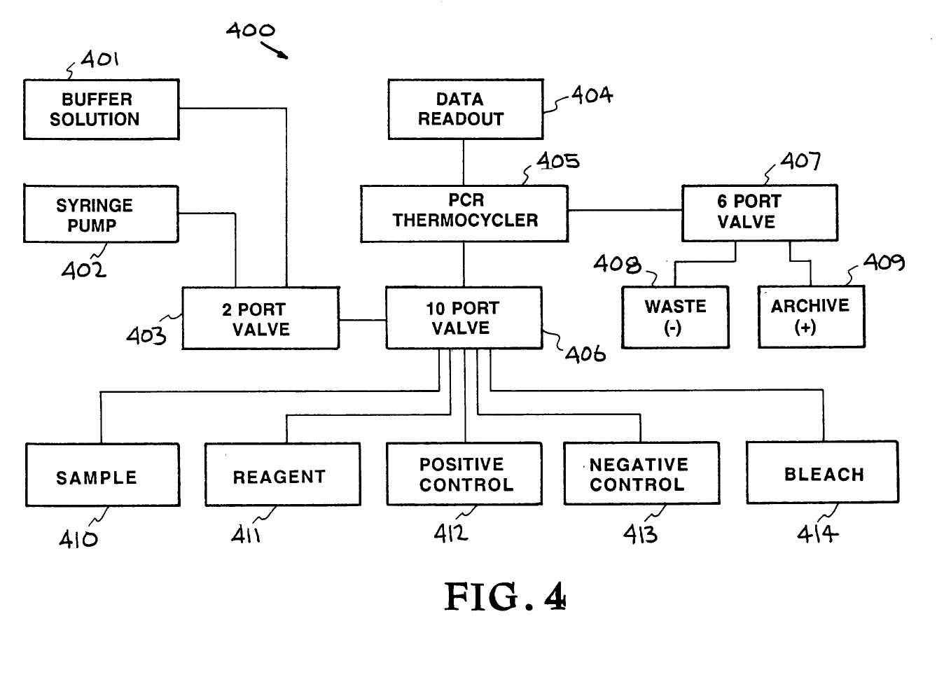

Referring now to FIG. 4, a system representing another embodiment of the present invention is illustrated. The system is generally designated by the reference numeral 400. In the system 400, carrier fluid 401 is drawn into a syringe pump 402 and then used to prime the mixing reactor and flow path connected to the central valve 406. Flow lines attached to sample 410, PCR reagents 411, and bleach reservoirs 414 are primed by sequentially drawing up fluid from each reservoir using the syringe pump 402.

Excess sample and reagent in the holding/mixing reactor is purged from the system 400 by flushing the system 400 with buffer 401 between each prime operation. Small volumes of separation medium (such as air or a hydrophobic liquid) are used to isolate aliquots of the sample and PCR reagents in the mixing/holding reactor. The isolated sample/PCR reagent mix is then transferred from the mixing/holding reactor into a silicon-based rapid thermocycler 405. The thermocycler 405 in addition to thermal control and detection elements, contains optical windows for delivering and detecting light. PCR amplification devices are described in publications such as U.S. Pat. No. 5,589,136 for silicon-based sleeve devices for chemical reactions, assigned to the Regents of the University of California, inventors: M. Allen Northrup, Raymond P. Mariella, Jr., Anthony V. Carrano, and Joseph W. Balch, patented Dec. 31, 1996 and many are commercially available such as ABI PRISM® 7700 Sequence Detection System by Applied Biosystems; iCycler iQ Real-Time PCR Detection System by Bio-Rad; and Smart Cycler® System by Cepheid.

As amplification occurs, real time detection of fluorescence-labeled TaqMan type probes occurs. Following amplification, the system 400 is decontaminated by flushing the thermocycler 405 and exposed flow lines with bleach 414. Heating the thermocycler chamber within thermocycler 405 in the presence of bleach is an additional step that is very effective in removing amplified PCR product. Any remaining bleach residue that could inhibit subsequent PCR amplifications is then removed by flushing the exposed flow lines with buffer solution. The system is then ready to perform the next sample preparation/detection operation.

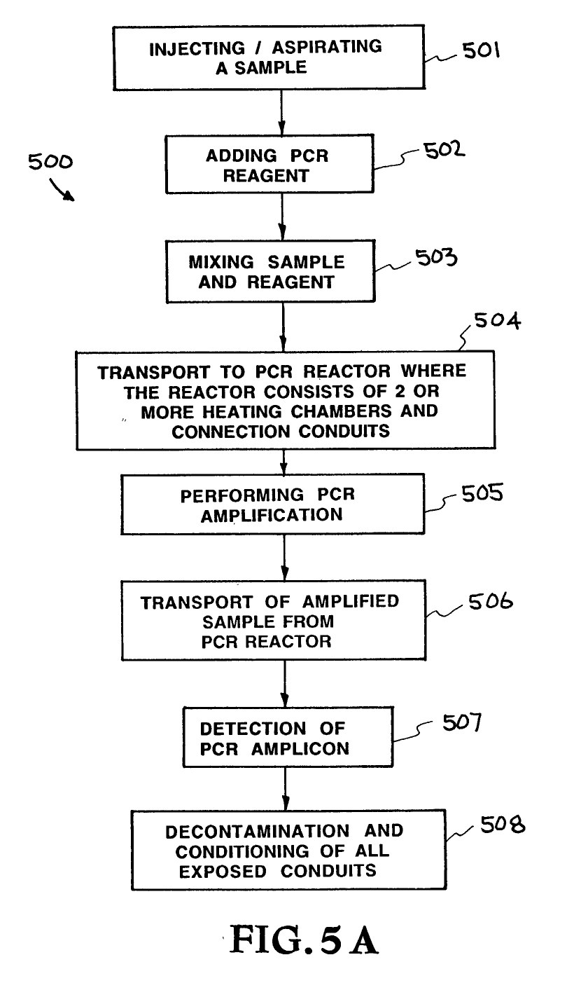

Referring now to FIG. 5A, another embodiment of a system for performing autonomous, nucleic acid assay is illustrated. The system is generally designated by the reference numeral 500. The system 500 provides a system capable of performing, singly or in combination, sample preparation, nucleic acid amplification, and nucleic acid detection functions. Some of the uses of the nucleic acid assay system 500 are: biowarfare detection applications including identifying, detecting, and monitoring bio-threat agents that contain nucleic acid signatures, such as spores, bacteria, etc.; biomedical applications including tracking, identifying, and monitoring outbreaks of infectious disease and automated processing, amplification, and detection of host or microbial DNA in biological fluids for medical purposes; forensic applications including automated processing, amplification, and detection DNA in biological fluids for forensic purposes; and food and beverage safety including automated food testing for bacterial contamination.

The nucleic acid assay system 500 includes a number components. A means 501 for injecting and or aspirating a sample provides injection and/or aspiration of the sample. In one embodiment the injecting/aspirating means 501 consists of a zone fluidics system. In another embodiment the injecting/aspirating means 501 consists of an FIA system. A means 502 for adding PCR reagent to the sample is operatively connected to the means 501 for injecting and or aspirating a sample. The components 501 through 504 can be, for example, units such as those contained in a single zone fluidics system called the FloPro-4P produced by Global FIA, Inc, Fox Island, Wash.

A means 503 for mixing the sample and the reagent is operatively connected to the means 502 for adding PCR reagent to the sample. The mixing means 503 mixes the sample with a PCR reagent. In one embodiment the PCR reagent includes primers. In another embodiment the PCR reagent includes oligos. A means 504 for transporting the sample and the reagent to a PCR reactor is operatively connected to the means 503 for mixing the sample and the reagent. The means 504 for transporting the sample and the reagent to a PCR reactor consists of a fluidics system. The means 504 for transporting the sample and the reagent to a PCR reactor consists of two or more heating chambers and a connection of conduits. Components 501 through 504 can be units, for example, units contained in a single zone fluidics system called the FloPro-4P produced by Global FIA, Inc, Fox Island, Wash.

A means 505 for performing PCR amplification is operatively connected to the means 504 for transporting the sample and the reagent to a PCR reactor. This results in an amplified sample. In one embodiment the PCR amplification means 505 includes an embedded thermocouple calibration conduit. PCR amplification devices are described in publications such as U.S. Pat. No. 5,589,136 for silicon-based sleeve devices for chemical reactions, assigned to the Regents of the University of California, inventors: M. Allen Northrup, Raymond P. Mariella, Jr., Anthony V. Carrano, and Joseph W. Balch, patented Dec. 31, 1996 and many are commercially available such as ABI PRISM® 7700 Sequence Detection System by Applied Biosystems; iCycler iQ Real-Time PCR Detection System by Bio-Rad; and Smart Cycler® System by Cepheid.

A means 506 for transporting the amplified sample from the PCR reactor is operatively connected to the means 505 for performing PCR amplification. The means 506 for transporting the amplified sample from the PCR reactor can be, for example, FEP tubing available from Cole-Parmer, Vernon Hills, Ill.

A means 507 for detection of PCR amplicon is operatively connected to the means 506 for transporting the amplified sample from the PCR reactor. The detector can be, for example, a detection system described in publications and products produced by Cepheid and Baltimore-based Environmental Technologies Group, Inc. (ETG), a part of London-based Smiths Aerospace.

Conduits are included within the means 501 for injecting and or aspirating a sample, means 502 for adding PCR reagent to the sample, means 503 for mixing the sample and the reagent, means 504 for transporting the sample and the reagent to a PCR reactor, means 505 for performing PCR amplification, means 506 for transporting the amplified sample from the PCR reactor, and means 507 for detection of PCR amplicon. A means 508 for decontamination and conditioning the conduits is directly connected to the means 507 for detection of PCR amplicon. The means 508 for decontamination and conditioning the conduits is operatively connected to the means 501 for injecting and or aspirating a sample, means 502 for adding PCR reagent to the sample, means 503 for mixing the sample and the reagent, means 504 for transporting the sample and the reagent to a PCR reactor, means 505 for performing PCR amplification, means 506 for transporting the amplified sample from the PCR reactor, and means 507 for detection of PCR amplicon. The means 508 for decontamination and conditioning of all exposed conduits can be accomplished by a decontaminant, such as bleach, being pumped through the exposed conduits and then washed from the system with a suitable wash solution.

Referring now to FIG. 5B another embodiment of a system for performing autonomous, in-line nucleic acid sample preparation, amplification, and/or analysis is illustrated. The system is generally designated by the reference numeral 550. The system 550 illustrates another embodiment of an amplification cell. The system 550 is an amplification system that is coupled to units such as units 151, 502, 503, and 504 of FIG. 5A. The system 550 includes a means for performing PCR amplification 551 and a means for detection of PCR amplicon 552 operatively connected to the means for performing PCR amplification 551. The detection is performed within the PCR reactor. The system 550 results in an amplified sample and detection of PCR amplicon is performed on the amplified sample. In one embodiment the PCR amplification means 551 includes an embedded thermocouple calibration conduit.

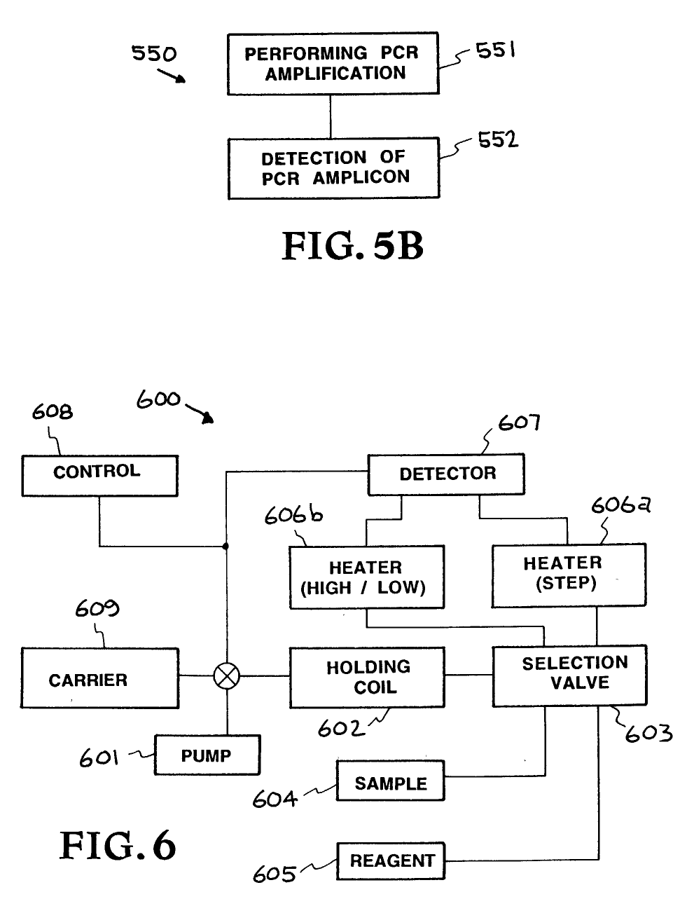

Referring now to FIG. 6 yet another embodiment of a system for performing autonomous, nucleic acid assay is illustrated. The system is generally designated by the reference numeral 600. The system 600 provides a system capable of performing, singly or in combination, sample preparation, nucleic acid amplification, and nucleic acid detection functions. Some of the uses of the nucleic acid assay system 600 are: biowarfare detection applications including identifying, detecting, and monitoring bio-threat agents that contain nucleic acid signatures, such as spores, bacteria, etc.; biomedical applications including tracking, identifying, and monitoring outbreaks of infectious disease and automated processing, amplification, and detection of host or microbial DNA in biological fluids for medical purposes; forensic applications including automated processing, amplification, and detection DNA in biological fluids for forensic purposes; and food and beverage safety including automated food testing for bacterial contamination.

The computer controlled system 600 performs sample preparation, sample delivery, sample isolation, and system decontamination functions. It is to be understood that multiple embodiments are envisioned. In the system 600, sample preparation and delivery is accomplished using a system including a pump 601, holding coil 602, selector valve 603, sample reservoir 604, and reagent reservoir 605. A heater (High/Low) 606A and heater (Step) 606B, and detector 367 are connected to the selection valve 603. A control unit 608 is operatively connected to the selection valve 603, the valve 610, pump 601, heater (High/Low) 606A and heater (Step) 606B, and detector 607. The control unit 608 and be a multipurpose computer or an individual control unit.

The pump 601 is used to draw and pump fluids into the holding coil 602. Fluids can be drawn from the sample unit 604 and the reagent unit 605. The carrier fluid unit 609 provides the medium for translating the pump movements into fluid handling actions. Aliquots of air or a hydrophobic liquid are used to spatially separate the carrier from reagent and sample volumes, greatly minimizing the chance of cross-contamination. The performance characteristics of the pump 601 allow for precise and accurate metering and positioning of aspirated zones in the flow manifold and flow cell. The holding coil 602 serves to mix various assay components (i.e., sample, oligonucleotides, primer, TaqMan probe, etc.) in preparation for amplification and/or detection. The holding coil 602 prevents contamination and is itself easily decontaminated by rinsing with buffer or some other cleaning agent (e.g., bleach). Nucleic acid amplification takes place in the heater (High/Low) 606A and heater (Step) 606B once the pump 601 and control 608 have positioned the relevant components in the heater (High/Low) 606A and heater (Step) 606B. Nucleic acid detection and analysis takes place in the detector 607 once the pump 601 and control 608 have positioned the relevant components in the detector 607. The selection valve 603 serves as the interface between all components of the unit, offering a flexible means of changing and upgrading the various fluidic components.

The nucleic acid amplification heater (High/Low) 606A and heater (Step) 606B performs in-line amplification of the target DNA. This amplification is typically achieved using polymerase chain reaction (PCR) based methodologies, where a prepared sample and reagent mix is isolated and thermal cycling performed. This repeated heating and cooling of the mix selectively doubles a nucleic acid sequence during each thermal cycle. This process can occur in any thermal cycling type device that is amenable to PCR type amplification, including rapid micro-machined silicon type cyclers, block heater-based cyclers, etc. such as those designed by Idaho Tech. systems that use isothermic, enzyme regulated amplification can also be used.

Detection is detector 607 can occur either during (i.e., “real-time”) or after the amplification process. Real-time detection of amplified nucleic acid sequences is often preferable in field applications, because it does not require time-consuming post-PCR manipulation and processing. Examples of such post-PCR processes include slab gel and capillary electrophoresis, hybridization to immobilized oligonucleotides, or mass spectrometry. Real-time PCR can be accomplished using optical-based assays that either increase or decrease the emission from fluorescence-labeled probes during each amplification step. One commonly used technique for real-time PCR is TaqMan, a homogeneous PCR test that uses a fluorescence resonance energy transfer probe. This probe typically contains a “reporter” dye at the 5′ end and a “quencher” dye at the 3′ end. Intact, there is very little fluorescent emission from the probe, since the proximity of the quencher to the reporter dye serves to suppress the reporter emission. During PCR amplification, the probe anneals to a targeted complementary amplicon strand and begins extending one of the primers. An enzyme, (Taq polymerase) cleaves the probe and displaces both dye molecules, allowing them to separate and diffuse into the surrounding fluid. The resulting increase in reporter emission can be monitored and correlated PCR product concentration.

The systems described above provide a nucleic acid assay system for analyzing a sample using a reagent. A holding means is provided for receiving the sample and the reagent. A PCR reactor means is provided for amplifying the sample. A detection means is provided for detection of PCR amplicon. A transport means is provided for selectively transporting the sample and the reagent to the holding means, the PCR reactor means, and the detection means. The transport means is operatively connected to the holding means, the PCR reactor means, and the detection means. A control means is provided for selectively adding the reagent to the sample, mixing the sample and the reagent, performing PCR amplification, and detecting PCR amplicon. The control means is operatively connected to the holding means, the PCR reactor means, the detection means, and the transport means. A decontamination means is provided for decontaminating the holding means, the PCR reactor means, and the detection means.

Conduits are included within the holding means, the PCR reactor means, the detection means, and the transport means. In one embodiment the conduits include tubing. In one embodiment the conduits include microchannels. In one embodiment the conduits include passages within the PCR reactor means. The decontamination means includes means for decontaminating the conduits.

The holding means mixes the sample with the reagent. In one embodiment the reagent is a PCR reagent. In one embodiment the PCR reagent includes primers. In one embodiment the PCR reagent includes oligos. In one embodiment the PCR reagent includes enzymes.

In one embodiment the PCR reactor means cycles between a relatively high temperature and a relatively low temperature to produce PCR amplification. In one embodiment the PCR reactor means includes a section that can be held at a relatively high temperature and a section that can be held at a relatively low temperature and the PCR reactor means cycles the sample between the section that can be held at a relatively high temperature and the section that can be held at a relatively low temperature. In one embodiment the PCR reactor means includes an embedded thermocouple calibration conduit.

The systems described above provide a nucleic acid assay method for analyzing a sample using a reagent. A holding means is provided for receiving the sample and the reagent, A PCR reactor means is provided for amplifying the sample. A detection means is provided for detection of PCR amplicon. The sample and the reagent are transported to the holding means, the PCR reactor means, and the detection means. The transport means is operatively connected to the holding means, the PCR reactor means, and the detection means. A decontamination means is provided for decontaminating the holding means, the PCR reactor means, and the detection means. A control means is provided for selectively mixing the sample and the reagent, performing PCR amplification, detecting PCR amplicon, and decontaminating the holding means, the PCR reactor means, and the detection means. The control means is operatively connected to the holding means, the PCR reactor means, and the decontamination means.

The PCR reactor means in one embodiment includes a section that can be held at a relatively high temperature and a section that can be held at a relatively low temperature. The PCR reactor means cycles the sample between the section that can be held at a relatively high temperature and the section that can be held at a relatively low temperature.

The systems described above have many uses including the following: biowarfare detection applications including identifying, detecting, and monitoring bio-threat agents that contain nucleic acid signatures, such as spores, bacteria, etc.; biomedical applications including tracking, identifying, and monitoring outbreaks of infectious disease, automated processing, amplification, and detection of host or microbial DNA in biological fluids for medical purposes; forensic applications including automated processing, amplification, and detection of DNA in biological fluids for forensic purposes; and food and beverage safety including automated food testing for bacterial contamination.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A nucleic acid assay system for analyzing a sample using a reagent, comprising:
holding means for receiving said sample and said reagent;
PCR reactor means for amplifying said sample;
detection means for detection of PCR amplicon;
transport means for selectively transporting said sample and said reagent to said holding means, said PCR reactor means, and said detection means, said transport means operatively connected to said holding means, said PCR reactor means, and said detection means;
control means for selectively adding said reagent to said sample, mixing said sample and said reagent, performing PCR amplification, and detecting PCR amplicon, said control means operatively connected to said holding means, said PCR reactor means, said detection means, and said transport means; and
means for decontaminating said holding means, said PCR reactor means, said detection means.

2. The nucleic acid assay system of claim 1, including conduits within said holding means, said PCR reactor means, said detection means, and said transport means; and wherein said means for decontaminating said holding means, said PCR reactor means, said detection means includes means for decontaminating said conduits.

3. The nucleic acid assay system of claim 2, wherein said conduits include tubing.

4. The nucleic acid assay system of claim 2, wherein said conduits include microchannels.

5. The nucleic acid assay system of claim 2, wherein said conduits include passages within said PCR reactor means.

6. The nucleic acid assay system of claim 1, wherein said holding means mixes said sample with said reagent.

7. The nucleic acid assay system of claim 6, wherein said reagent is a PCR reagent.

8. The nucleic acid assay system of claim 6, wherein said PCR reagent includes primers.

9. The nucleic acid assay system of claim 6, wherein said PCR reagent includes oligos.

10. The nucleic acid assay system of claim 6, wherein said PCR reagent includes enzymes.

11. The nucleic acid assay system of claim 1, wherein said PCR reactor means cycles between a relatively high temperature and a relatively low temperature to produce PCR amplification.

12. The nucleic acid assay system of claim 1, wherein said PCR reactor means includes a section that can be held at a relatively high temperature and a section that can be held at a relatively low temperature and said PCR reactor means cycles said sample between said section that can be held at a relatively high temperature and said section that can be held at a relatively low temperature.

13. The nucleic acid assay system of claim 1, wherein said PCR reactor means includes an embedded thermocouple calibration conduit.

14. A nucleic acid assay method for analyzing a sample using a reagent, comprising the steps of:
providing a holding means for receiving said sample and said reagent;
providing a PCR reactor means for amplifying said sample;
providing a detection means for detection of PCR amplicon;
transporting said sample and said reagent to said holding means, said PCR reactor means, and said detection means; said transport means operatively connected to said holding means, said PCR reactor means, and said detection means;
providing a decontamination means for decontaminating said holding means, said PCR reactor means, said detection means; and
providing a control means for selectively mixing said sample and said reagent, performing PCR amplification, detecting PCR amplicon, and decontaminating said holding means, said PCR reactor means, and said detection means; said control means operatively connected to said holding means, said PCR reactor means, and said decontamination means.

15. The nucleic acid assay method of claim 14, including conduits within said holding means, said PCR reactor means, said detection means, and said transport means; and wherein said decontamination means includes means for decontaminating said conduits.

16. The nucleic acid assay method of claim 15, wherein said conduits include tubing.

17. The nucleic acid assay method of claim 15, wherein said conduits include microchannels.

18. The nucleic acid assay method of claim 15, wherein said conduits include passages within said PCR reactor means.

19. The nucleic acid assay method of claim 14, wherein said sample and said reagent are mixed within said holding means.

20. The nucleic acid assay method of claim 19, wherein said reagent is a PCR reagent.

21. The nucleic acid assay method of claim 20, wherein said PCR reagent includes primers.

22. The nucleic acid assay method of claim 20, wherein said PCR reagent includes oligos.

23. The nucleic acid assay system of claim 20, wherein said PCR reagent includes enzymes.

24. The nucleic acid assay system of claim 14, including the step of cycling said sample between a relatively high temperature and a relatively low temperature to produce PCR amplification.

25. The nucleic acid assay system of claim 14, wherein said PCR reactor means includes a section that can be held at a relatively high temperature and a section that can be held at a relatively low temperature and said PCR reactor means cycles said sample between said section that can be held at a relatively high temperature and said section that can be held at a relatively low temperature.

26. A nucleic acid assay method for analyzing a sample, comprising the steps of:
utilizing a holding vessel for mixing said sample with a reagent;
utilizing a reactor for amplifying said sample and producing an amplified sample;
utilizing a detector for detecting PCR amplicon;
utilizing a fluidic system for selectively transporting said sample, said reagent, and said amplified sample relative to said holding means;
decontaminating and conditioning said nucleic acid assay system; and
utilizing a control for controlling the selectively adding of said reagent to said sample, mixing of said sample and said reagent, performing PCR amplification, detecting PCR amplicon, and decontaminating and conditioning said nucleic acid assay system.

27. The nucleic acid assay method of claim 26, wherein said reagent is a PCR reagent.

28. The nucleic acid assay method of claim 26, wherein said PCR reagent includes primers.

29. The nucleic acid assay method of claim 26, wherein said PCR reagent includes oligos.

30. The nucleic acid assay system of claim 26, wherein said PCR reagent includes enzymes.

31. The nucleic acid assay system of claim 26, including the step of cycling said sample between a relatively high temperature and a relatively low temperature to produce PCR amplification.
